(19) United States
(12) Patent Application Publication
(10) Pub. No.: US 2021/0330419 A1
(43) Pub. Date: Oct. 28, 2021

(54) PATIENT BARRIER DEVICE AND ASSOCIATED SYSTEM AND METHOD FOR REDUCING EXPOSURE BETWEEN A PATIENT AND CAREGIVER

(71) Applicant: DR. J.L. DANNER, INC., Canton, OH (US)

(72) Inventor: Jeffrey L. Danner, Canton, OH (US)

(21) Appl. No.: 17/240,388

(22) Filed: Apr. 26, 2021

Related U.S. Application Data
(60) Provisional application No. 63/014,770, filed on Apr. 24, 2020.

(51) Int. Cl. A61B 90/40; A61B 90/50

(52) U.S. Cl. CPC A61B 90/40; A61B 2090/401; A61B 2217/005; A61B 90/50

(57) ABSTRACT

A patient barrier device includes a shield that defines a protective volume subjacent the shield and a patient's face. The patient barrier device further includes a fluid port in fluid communication with the protective volume. The patient barrier device further includes a mounting feature connected to the shield for supporting the shield relative to the patient's face. A system includes a patient barrier device having a shield that defines a protective volume subjacent the shield and the patient's face. The system further includes a pump coupled with the patient barrier device. A method includes positioning a patient barrier device over a face of a patient, the patient barrier device having a shield defining a protective volume over the face of the patient. The method further includes drawing air from the protective volume while the face of the patient is in the protective volume.

### PRIORITY

This application claims priority from U.S. Ser. No. 63/014,770 filed on Apr. 24, 2020.

### FIELD

The present application relates to equipment used during medical and dental procedures and, more specifically, to a device, system, and method for protecting caregivers from exposure to airborne or infectious diseases deriving from a patient.

### BACKGROUND

Personal protective equipment (PPE) in medical and dental caregivers is important for the providers' protection. Further, PPE is critical in prevention of cross-contamination with other patients in the vicinity. To achieve protection for caregivers, such as doctors, physicians, dentists, nurses, and EMTs, it is typical for providers to wear various levels of PPE. This protects the providers but does not help prevent cross-contamination to others in the vicinity.

Accordingly, those skilled in the art continue with research and development efforts in the field of PPE.

### SUMMARY

Disclosed is a patient barrier device.

In one aspect, the patient barrier device includes a shield that defines a protective volume subjacent the shield. The patient barrier device further includes a fluid port in fluid communication with the protective volume. The patient barrier device further includes a mounting feature connected to the shield for supporting the shield relative to the patient's face.

Also disclosed is a system for reducing exposure between a patient and a caregiver.

In one aspect, the system includes a patient barrier device having a shield that defines a protective volume subjacent the shield. The system further includes a pump coupled with the patient barrier device.

Also disclosed is a method for reducing exposure between a patient and a caregiver.

In one aspect, the method includes positioning a patient barrier device over a face of a patient, the patient barrier device having a shield defining a protective volume over the face of the patient. The method further includes drawing air from the protective volume while the face of the patient is in the protective volume.

### BRIEF DESCRIPTION OF THE DRAWINGS

FIG. 1 is a side view of a patient barrier device;

FIG. 2 is a side view of the patient barrier device of FIG. 1 in use by a patient;

FIG. 3 is a top view of the patient barrier device of FIG. 1;

FIG. 4 is a top view of the patient barrier device of FIG. 1, in use by a patient;

FIG. 5 is a front view of the patient barrier device of FIG. 1;

FIG. 6 is a side view of a structure;

FIG. 7 is a bottom view of the structure of FIG. 6;

FIG. 8 is a top view of the patient barrier device of FIG. 1 coupled with the structure of FIG. 6

FIG. 9 is a side view of the patient barrier device coupled with a different structure;

FIG. 10 is a top view of the patient barrier device coupled with the structure of FIG. 9;

FIG. 11 is a block diagram of a system for reducing exposure between a patient and a caregiver; and

FIG. 12 is a flowchart of a method for reducing exposure between a patient and a caregiver.

### DETAILED DESCRIPTION

The following detailed description refers to the accompanying drawings, which illustrate specific examples of the disclosed containers and closure apparatus. It will be understood that the disclosed examples are merely exemplary embodiments of the way in which certain aspects of the invention can be implemented and do not represent an exhaustive list of all the ways the invention may be embodied. Other examples having different structures and operations do not depart from the scope of the present disclosure. Like reference numerals may refer to the same feature, element, or component in the different drawings. The figures are not necessarily to scale and some features may be exaggerated or minimized to show details of particular components. Throughout the present disclosure, any one of a plurality of items may be referred to individually as the item and a plurality of items may be referred to collectively as the items. Moreover, as used herein, a feature, element, component, or step preceded with the word “a” or “an” should be understood as not excluding a plurality of features, elements, components, or steps, unless such exclusion is explicitly recited.

Illustrative, non-exhaustive examples, which may be, but are not necessarily, claimed, of the subject matter according to the present disclosure are provided below. Reference herein to “example” means that one or more feature, structure, element, component, characteristic, and/or operational step described 156 in connection with the example is included in at least one aspect, embodiment, and/or implementation of the subject matter according to the present disclosure. Thus, the phrases “an example,” “another example,” “one or more examples,” and similar language throughout the present disclosure may, but do not necessarily, refer to the same example. Further, the subject matter characterizing any one example may, but does not necessarily, include the subject matter characterizing any other example. Moreover, the subject matter characterizing any one example may be, but is not necessarily, combined with the subject matter characterizing any other example.

During medical and dental procedures, aerosols are created using rotary instrumentation, sonic instrumentation, and high-speed instrumentation with water cooling systems. These aerosols are created as the saliva, blood, and necrotic tissue are picked up by the rotary movement of the instrumentation. The importance of addressing the aerosols is that the mouth is connected to the nasal cavity and airway. This aerosol spray may consist of microscopic disease-causing components of bacteria, viruses, and fungi. As the aerosol spray leaves the mouth it jettisons the disease-causing droplets into the air requiring the need for containment for the protection of doctors, physicians, dentists, EMTs, hygienists, dental assistants, office personnel, patients, and visitors of the medical or dental office. This can cause contamination in the office because “[t]he results showed that bacterial aerosols contamination could spread a horizontal distance of 100 cm and a vertical distance of 50 cm from a patient's oral cavity, and remain airborne . . . ” Investigation of the Spreading Characteristics of Bacterial Aerosol Contamination During Dental Scaling Treatment, Chuang and Chuang, Journal of Dental Science, June 2014 Additionally, “large particles (5 to 15 micrometers) will not immediately drop to the ground but will remain airborne several minutes. Smaller particles (less than 5 micrometers) will remain in the air for many minutes or even hours.” Commentary: COVID-19 Transmission Message Should Hinge on Science, Brosseau, Center for Infectious Disease Research and Policy, University of Minnesota, March 2020.

The “first wave” of containment of these aerosols is typically done with various plastic intra oral devices used to isolate the area within the mouth being treated. High volume suction within this device and secondary high-volume suction at the specific site contains some of the spray.

The remaining spray will diffuse to the surrounding area, external to the patient's oral cavity, and needs to be controlled while still allowing the doctor, dentist, hygienist, or dental assistant to operate. Typically, this involves lab coats covering from neck to wrists where gloves cover the hands. Above the collar the caregiver typically wears a mask and eye protection. Thus, a large portion of the caregivers' heads can be exposed, as well as the entire procedure room.

To remedy this issue, a patient barrier device 10 fluidly coupled with a pump 150 or suction can appropriately cover the operating area of the patient while still allowing for the caregiver's ability to move with their hands in multiple directions, up and down and close to the patient and further out. With the patient barrier device 10, the aerosols can be contained within a protective volume 100 at their source, i.e., proximate the patient's mouth, thus protecting the caregiver and the entire room. The patient barrier device 10 can be lightweight and comfortable for the patient to wear for extended periods of time. Alternatively, the patient barrier device 10 can be mounted on a medical light 45, chair 154, table, bed 156, or any other structure located proximate the patient such that the patient barrier device 10 defines a protective volume proximate the patient's face without touching the patient.

In one example, the patient barrier device 10 includes a headpiece assembly 20. In one example, the headpiece assembly 20 has a visor style design. The headpiece assembly 20 includes a frame 20a. In one example, the frame 20a is configured to support the patient barrier device 10 on the patient's head. The frame 20a is stabilized or supported on the patient's head with first mounting arm 17 and second mounting arm 19 laterally opposed from each other. In another example, the frame 20a is supported on a bed 156 or chair 154 on either side of the patient's head. In one example, a visor portion 25 extends out and away from the patient's forehead, for example, 10 inches, and is coupled with shield 15.

In one example, shield 15 is substantially dome shaped and comprised of a polymeric material with an exemplary standard industry thickness. In another example, the shield 15 is transparent. The shield 15 may disposable and may further be recyclable such that it is easily changed and disposed of between patients. Similarly, the patient barrier device 10 can be made of lightweight and transparent flexible plastic similar to face shields with flexibility while still holding its shape. Further, the shield 15 may comprise a flexible polymeric material.

In one example, the patient barrier device 10 is attached to the frame 20a within a groove 22. In one example, groove 22 is located in the visor portion 25 and is secured by several fasteners 24 attaching to corresponding holes in the shield 15 at the connecting point and can extend, for example, about 12 inches to about 24 inches towards the patient's stomach, and for example 24 inches side to side. The abdominal extension of the patient barrier device 10 could be taped to a drape placed on the patient's chest extending from the stomach to the forehead with an opening for the face. This configuration yields approximately 10 inches of space for the caregiver to insert their hands under the patient barrier device 10 to operate on the patient, which is within a typically comfortable and ergonomic working space. It can be important for caregivers to have an ergonomic workspace because they are bent over a patient for long periods of time, which can stress their backs.

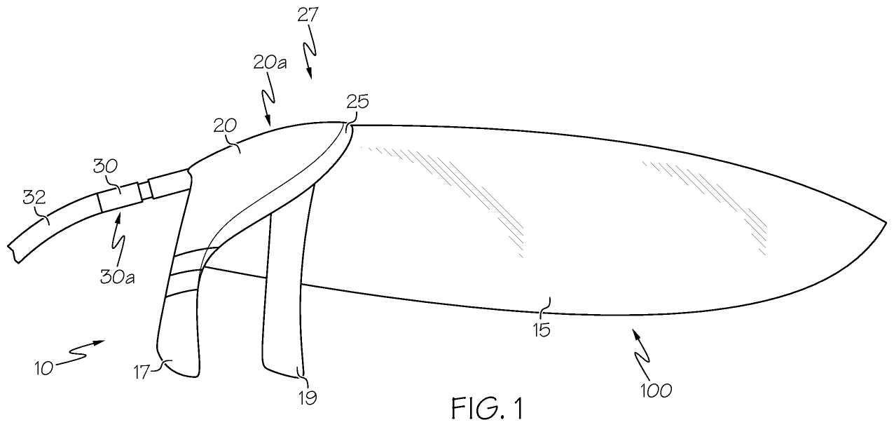

Referring to FIG. 1, disclosed is a patient barrier device 10. The patient barrier device 10 comprises a shield 15. The shield 15 defines a protective volume 100 subjacent the shield 15 and above a patient's face. The protective volume 100 defines an area in which a patient's expelled breath is contained, for example, proximate the patient's face and along the length and width of the shield 15. In one example, the shield 15 is substantially planar. In another example, the shield 15 is substantially dome shaped such that it has a curvature to define the protective volume 100.

Still referring to FIG. 1, the patient barrier device 10 includes a fluid port 30a in fluid communication with the protective volume 100. The fluid port 30a comprises a connector 30 for coupling the fluid port 30a with a pump 150. In one example, the pump 150 comprises a vacuum source 152. In one example, the patient barrier device 10 includes a second fluid port 30a in fluid communication with the protective volume 100.

Still referring to FIG. 1, the patient barrier device 10 includes a mounting feature 27 connected to the shield 15 for supporting the shield 15 relative to the patient's face. In one example, the mounting feature 27 includes a frame 20a configured to be supported on the patient's head with first mounting arm 17 and second mounting arm 19 laterally opposed from each other. In another example, the frame 20a is configured to be supported on a bed 156 or chair 154 on either side of the patient's head with first mounting arm 17 and second mounting arm 19. In yet another example, the mounting feature 27 comprises a fastener 44 for supporting the shield 15 on a structure 120.

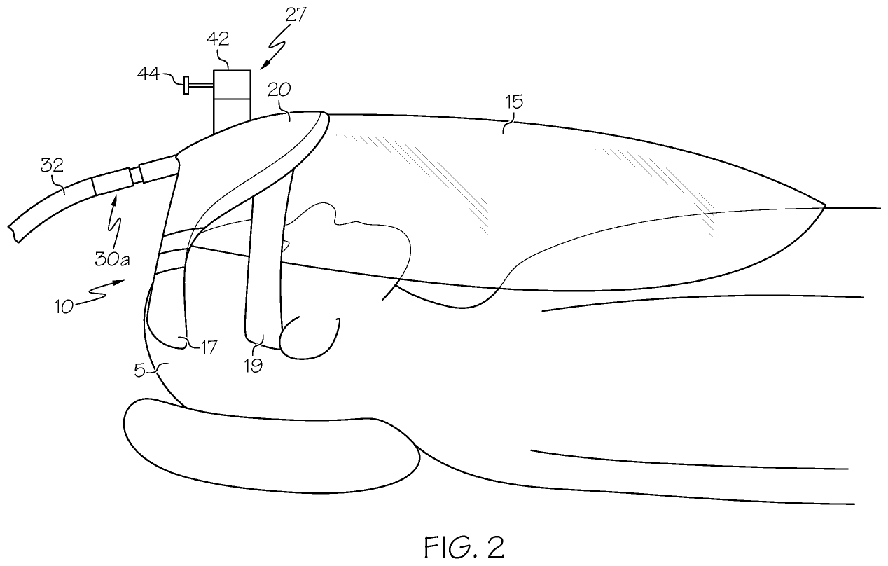

Referring to FIG. 2, in one example, mounting feature 27 includes an attachment mechanism 42 for coupling the patient barrier device 10 to a structure 120. The structure 120 may be any medical equipment in close proximity to the patient such as a medical or dental light 45, a medical or dental chair 154, a medical bed 156, a medical or dental mirror, or any other suitable means for positioning the patient barrier device 10 proximate the patient's face to define a protective volume 100.

In one example, the shield 15, fluid port 30a, and mounting feature 27 are integral such that the patient barrier device is a single, monolithic body. In another example, the shield 15 is removably coupled to the mounting feature 27.

Referring back to FIG. 1, illustrated is a side view of an example of the patient barrier device 10. The patient barrier device 10 includes a shield 15. In one example, the shield 15 is connected to a headpiece assembly 20. The patient barrier device 10 further includes a connector 30. In one example, the connector 30 includes a fluid port 30a for attaching a pump 150, such as a typical dental suction apparatus 151, or vacuum source 152. The headpiece assembly 20 can have straps or a similar means for holding the patient barrier device 10 in place with optimal patient comfort. Each connector 30 is a standard size connector to fit a standard medical or dental vacuum tubing assembly. Each connector 30 on the rear of the headpiece are operatively connected to each inlet 26 in the protective volume 100 defined by the patient barrier device 10 and the inner surface of the headpiece assembly 20. The air can travel through the inlets 26 of the headpiece assembly 20 and into suction tube 32. This provides a means by which aerosols coming from a patient's mouth can be safely removed from the protective volume 100 via suction at the patient's temporal area on each side and prevent fogging of the shield 15. Alternatively, the inlet 26 may be located substantially central in the headpiece assembly 20 such that the suction pulls from a central region of the protective volume 100.

FIG. 2 illustrates an example of the patient barrier device 10 in use with a patient. The headpiece assembly 20 has two of mounting feature 27, such as first mounting arm 17 and second mounting arm 19 on each side that can extend from the headpiece assembly 20 down each side of a patient's head. The first mounting feature 27, or first mounting arm 17 in FIG. 2, located most proximally to the connector 30, serves to stabilize the patient barrier device 10 as it rests on the side of the patient's skull. The second mounting feature 27, or second mounting arm 19, located under the patient barrier device 10, rests above the ears similarly as arms of a pair of glasses would rest. These two mounting features 27, or first mounting arm 17 and second mounting arm 19 may be detachable and are important for the stabilization of the patient barrier device 10 from right to left as the forehead contact of the headpiece assembly 20 supports horizontally. They can gently squeeze or “grab” the patient's head with a comfortable friction fit. Alternately, the mounting features 27, or first mounting arm 17 and second mounting arm 19 can rest on the chair 154 or pillow that is supporting the patient's head.

Still referring to FIG. 2, an example of attachment mechanism 42 is positioned centrally on top of the headpiece assembly 20. In one example, the attachment mechanism 42 is tubular in shape, and includes a fastener 44, for example, a thumbscrew. This attachment mechanism 42 can be useful for attaching the patient barrier device 10 to an overhead dental light 45 arm 40, a dental chair 154, medical chair 154, medical light, or any other structure in proximity to the patient's face.

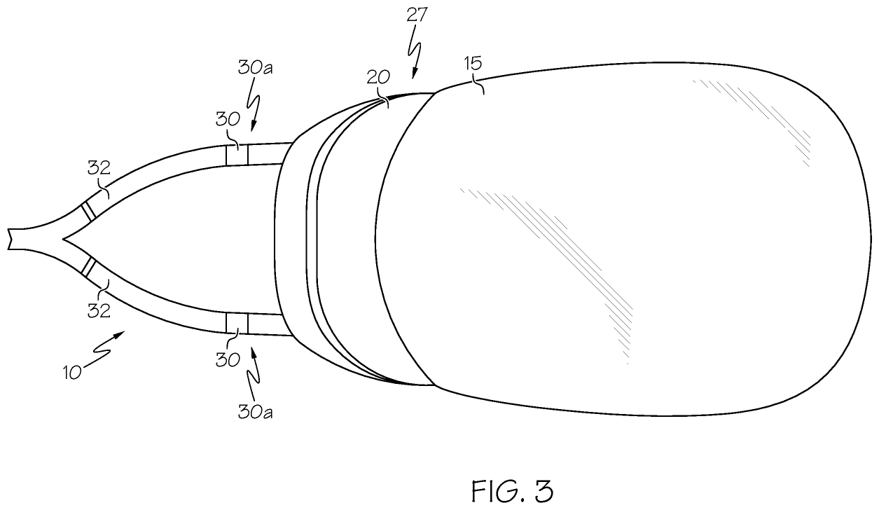

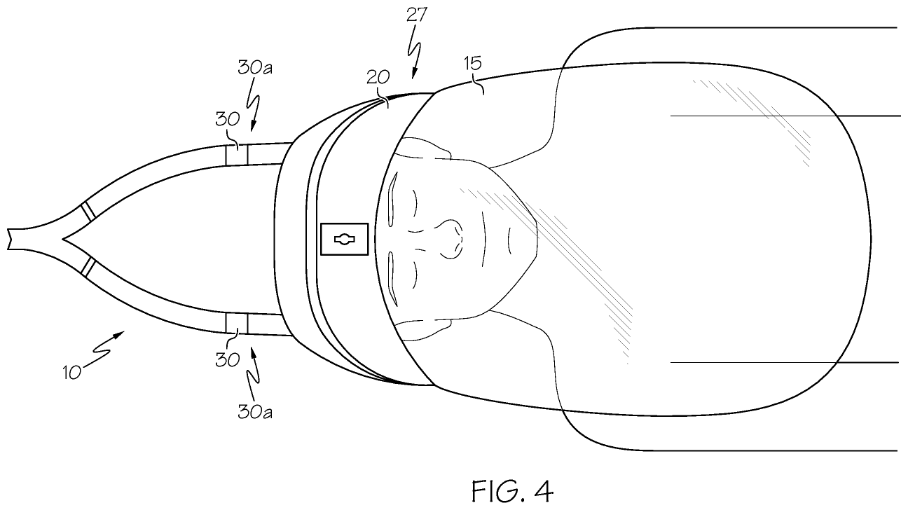

FIG. 3 illustrates a top view of the patient barrier device 10, and FIG. 4 illustrates a top view of an example of the patient barrier device 10 while being worn by a patient. The patient barrier device 10 extends beyond the patient's mouth by approximately 8 inches to 12 inches preferable approximately 10 inches. It can also extend to the right and left of the patient covering the patient's chest and shoulder. In FIGS. 3 and 4, each fluid port 30a can be seen at the right and left of the patient. By placing a fluid port 30a at the right and left, the patient barrier device 10 is substantially balanced. Alternatively, the fluid port 30a is located substantially central on the patient barrier device 10. Suction tubes 32 are coupled with each fluid port 30a via a connector 30 and can join, for example, at approximately 12-24 inches behind and below the patient's head and can further connect to the pump 150. In one example, the pump 150 is coupled with a caregiver's standard suction unit, such as a vacuum source 152. This obviates the need for a separate suction unit, saving space and cost.

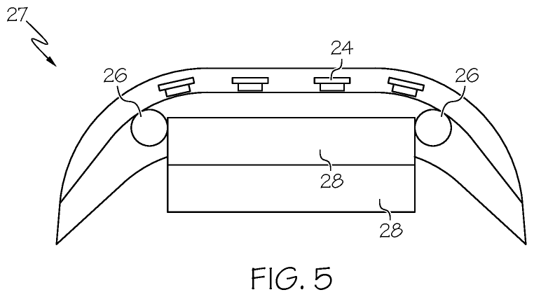

FIG. 5 illustrates headpiece assembly 20, viewing from the patient's feet toward the head, with the shield 15 removed. To accommodate different patients, the headpiece assembly 20 can have two or more removable forehead contact spacers 28 that can snap into an inner surface of the headpiece assembly 20 to allow proper horizontal ergonomic working space. These spacers 28 offer spacing between a patient's forehead and the shield 15. The headpiece assembly 20 also provides a groove 22 for the top edge of the shield 15 to fit into and attach. Fasteners 24, for example buttons, clips, or any similar means to allow removable attachment of the shield 15 to the headpiece assembly 20 are likewise provided. Each inlet 26 provided is operatively connected to a connector 30 on the rear of the headpiece assembly 20. Each inlet 26 can be positioned on both sides of the forehead spacers 28. Thus, air in the space confined by the patient barrier device 10 can be evacuated at the right and left inlets 26 at the temporal areas near the hairline 5. Each inlet 26 can be molded as an integral part of the headpiece assembly 20 such that it is a single, monolithic bod. In another example, each inlet 26 can likewise be formed by two separate pieces positioned inside the headpiece assembly 20. In this configuration, the separate pieces of tubing can be held in place by friction-fit brackets molded into the headpiece assembly 20.

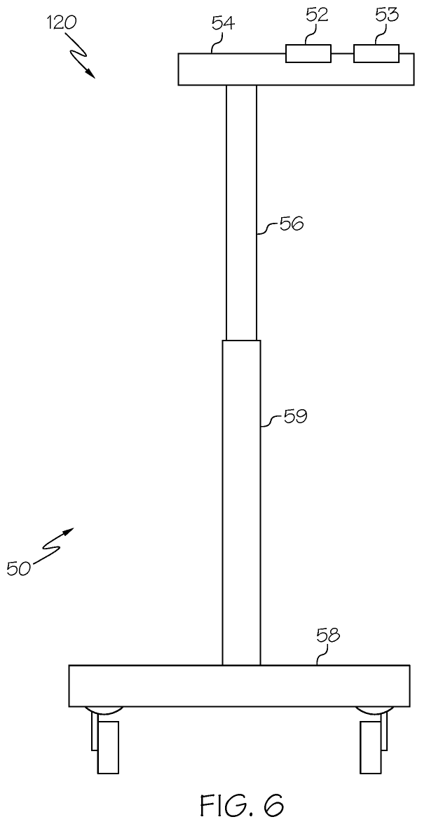

FIG. 6 illustrates a side view of a structure 120, for example a patient cart 50, which can be used to implement an example of the patient barrier device 10. The cart has two mounting structures 52 on each side of a U-shaped arm 54 that extends horizontally from a vertical pole 56. Vertical pole 56 can be slidably attached to base pole 59, which is attached to base 58. Vertical pole 56 can be inserted into or otherwise attached to base pole 59. Thus, the height of U-shaped arm 54 can be adjusted by sliding vertical pole 56 in or out of base pole 59. The patient barrier device 10 can attach to the arm via the two mounting structures 52 on each side for stability and optionally fastened into place with a standard fastener such as a screw, clip, or the like. This example can be useful in situations when the patient cannot be relied on for stability of the headpiece due to patient sedation or other limitations.

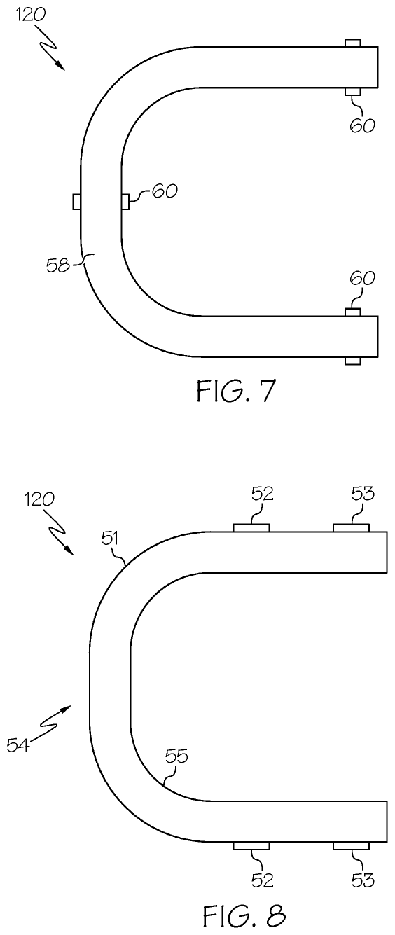

FIG. 7 illustrates an example of wheels 60 on the base 58 of the cart 50. The wheels 60 can be of a swivel type, such that they can easily guide the cart 50 in any direction. This configuration of wheels 60 allows for stability and ease of adjustment at the head of the patient chair 154 as may be needed with patient movement.

FIG. 8 illustrates a top view of a structure 120. In one example, the arm 54 of the cart 50 can serve as a stabilizing means for the patient barrier device 10. mounting structures 52 are shown as positioned on the outside surface 51 of arm 54. Mounting structures 52 can also be positioned on the inside surface 55 of arm 54. The patient barrier device 10 can attach to the arm 54 by inserting first mounting arm 17 and second mounting arm 19 on the headpiece assembly 20 into the mounting structures 52. For example, first mounting arm 17 can be inserted into one mounting structure 52 and second mounting arm 19 can be inserted into another mounting structure 52 on each side of the U-shaped arm 54 for a total of four insertions. The first mounting arm 17 and second mounting arm 19 can be optionally secured into place with a standard fastener such as a screw, clip, or the like. With support in both vertical and horizontal dimensions, the patient barrier device 10 can be easily moved by adjusting the cart 50 with wheels 60.

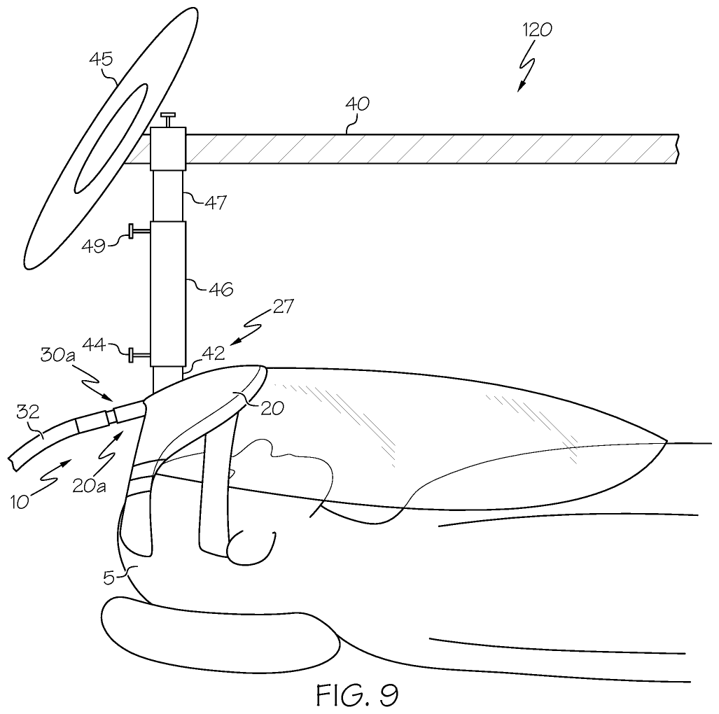

FIG. 9 illustrates a side view of an example of the patient barrier device 10 mounted to a structure 120 via mounting feature 27. An existing overhead dental light 45 arm 40 can provide stabilization of the patient barrier device 10 without patient head position limitations. Dental light 45 is also attached to the apparatus arm 40. Arm 40 is shown as a component of a dental light 45, but alternately, arm 40 could be an auxiliary arm that is a component of a medical or dental unit. The patient barrier device 10 can be attached to the dental light 45 arm 40 via a vertical bar 46 that is slidably attached to inner bar 47, which is attached to and descends from the horizontal arm 40 as shown. The descending bar 46 can be inserted into or otherwise attached to the attachment mechanism 42 fixed to the headpiece assembly 20, and can be secured by a fastener 44, for example, a thumbscrew. Additionally, the distance from arm 40 to patient barrier device 10 can be adjustable, for example, by sliding inner bar 47 in or out of vertical bar 46. The length can be adjustably fixed by fastener 49, for example, a thumbscrew. Thus, the patient barrier device 10 can be removably and adjustably attached to the overhead dental light 45 arm 40 and can be easily moved by using a handle attached to the overhead dental light 45. This third exemplary embodiment would obviate the need for a separate cart for holding the patient barrier device 10, thus providing a cost savings, and can be useful in situations when the patient cannot be relied on for stability of the patient barrier device 10 due to patient sedation or other limitations.

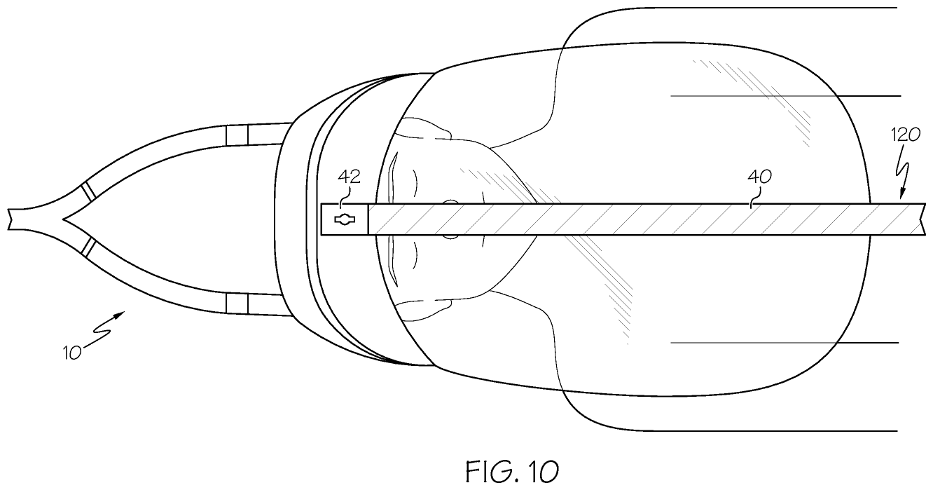

FIG. 10 illustrates a top view of the horizontal arm 40 attached to the patient barrier device 10 in use with a patient. In one example, the horizontal arm 40 is pivotably coupled with the patient barrier device 10 such that it may extend along the patient's torso as illustrated in FIG. 10 or, alternatively, approximately 90 degrees from that configuration, or any other desired angle.

In one example, the patient barrier device 10 serves as an aerosol mitigation device comprising a transparent shield 15 removably attached to a front side of a headpiece assembly 20. The headpiece assembly 20 has an inner surface and an outer surface. The headpiece assembly 20 further has at least one connector 30 positioned on a rear side of the headpiece assembly 20, and at least one inlet 26 positioned on the inner surface of the headpiece assembly 20. The at least one connector 30 is operatively connected to the at least one inlet 26.

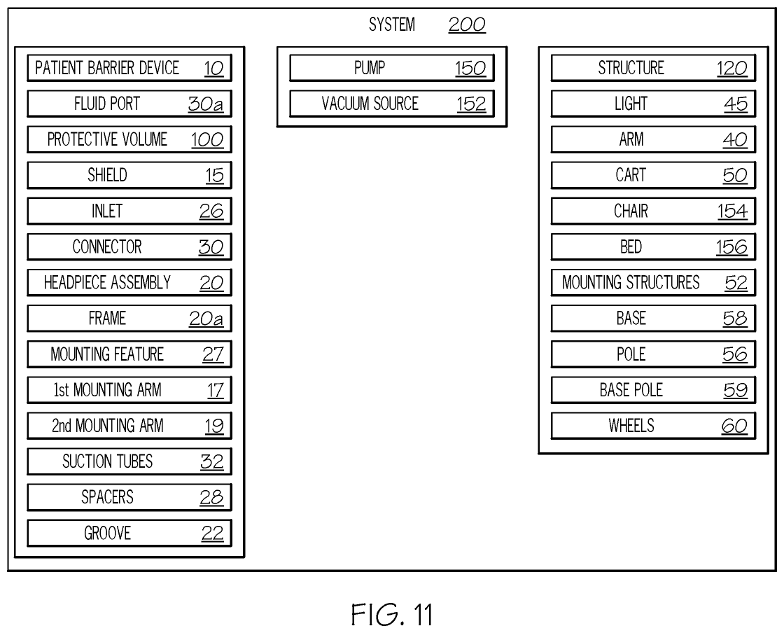

Referring to FIG. 11, disclosed is a system 200 for reducing exposure between a patient and a caregiver. In one example, the system 200 comprises a patient barrier device 10 comprising a shield 15 that defines a protective volume 100 subjacent the shield 15 and above the patient's face. The system 200 further comprises a pump 150 coupled with the patient barrier device.

Still referring to FIG. 11, in one example, the patient barrier device 10 of system 200 comprises a fluid port 30a in fluid communication with the protective volume 100 and the pump 150. In another example, the patient barrier device 10 comprises a second fluid port 30a in fluid communication with the protective volume 100 and the pump 150. The pump 150 may include a vacuum source 152 configured to pull air from the protective volume 100 and filter it to remove any contagious particulate in the air.

Still referring to FIG. 11, the patient barrier device 10 comprises a structure 120 for supporting the shield 15 over the patient's face. In one example, the structure 120 is a medical or dental light. In another example, the structure 120 is a medical or dental chair 154. In yet another example, the structure 120 is a medical bed 156. Further, the structure 120 may comprise any medical equipment within proximity of the patient.

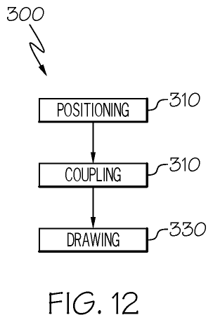

Referring to FIG. 12, a method 300 for reducing exposure between a patient and a caregiver is disclosed. In one example, the method 300 includes positioning 310 a patient barrier device 10 over a face of a patient. The patient barrier device 10 comprises a shield 15 defining a protective volume 100 over the face of the patient. The patient barrier device 10 includes a fluid port 30a in fluid communication with the protective volume 100. The fluid port 30a comprises a connector 30 for coupling the fluid port 30a with a pump 150.

Still referring to FIG. 12, the method 300 further includes coupling 320 a vacuum source 152 with the patient barrier device 10. The vacuum source 152 is configured to pull air from the protective volume 100 and may further transfer that air to a filtration apparatus for further sterilization. The coupling 320 is achieved by coupling the vacuum source 152 with the patient barrier device 10 via a fluid port 30a for the transfer of air from the protective volume 100 to the vacuum source 152.

Still referring to FIG. 12, the method 300 further includes drawing 330 air from the protective volume 100 while the face of the patient is in the protective volume 100. The air may be drawn with a pump 150, and further for example a vacuum source 152. I one example, the air travels through inlets 26 of the patient barrier device 10 and into one or more suction tube 32.

Although various examples of the disclosed container and closure apparatus have been shown and described 156, modifications may occur to those skilled in the art upon reading the specification. The present application includes such modifications and is limited only by the scope of the claims.

What is claimed is:

1. A patient barrier device comprising:
a shield that defines a protective volume subjacent the shield;
a fluid port in fluid communication with the protective volume; and
a mounting feature connected to the shield for supporting the shield relative to the patient's face.

2. The patient barrier device of claim 1, wherein the shield, fluid port, and mounting feature are integral such that the patient barrier device is a single, monolithic body.

3. The patient barrier device of claim 1, wherein the shield is removably coupled to the mounting feature.

4. The patient barrier device of claim 1, wherein the shield comprises a flexible polymeric material.

5. The patient barrier device of claim 1, wherein the shield comprises a transparent material.

6. The patient barrier device of claim 1, wherein the shield is recyclable.

7. The patient barrier device of claim 1, wherein the mounting feature comprises a frame for supporting the shield on the patient's face.

8. The patient barrier device of claim 1, wherein the mounting feature comprises a fastener for supporting the shield on a structure.

9. The patient barrier device of claim 8, wherein the structure comprises a medical light.

10. The patient barrier device of claim 8, wherein the structure comprises a medical chair.

11. The patient barrier device of claim 1, further comprising a second fluid port in fluid communication with the protective volume.

12. The patient barrier device of claim 1, wherein the fluid port is coupled with a pump.

13. A system for reducing exposure between a patient and a caregiver, the system comprising:
a patient barrier device comprising a shield that defines a protective volume subjacent the shield; and
a pump coupled with the patient barrier device.

14. The system of claim 13, wherein the patient barrier device comprises a fluid port in fluid communication with the protective volume and the pump.

15. The system of claim 14, wherein the patient barrier device comprises a second fluid port in fluid communication with the protective volume and the pump.

16. The system of claim 13, wherein the pump comprises a vacuum source.

17. The system of claim 13, further comprising a structure for supporting the shield over the patient's face.

18. The system of claim 17, wherein the structure comprises medical equipment.

19. A method for reducing exposure between a patient and a caregiver, the method comprising:
positioning a patient barrier device over a face of a patient, the patient barrier device comprises a shield defining a protective volume over the face of the patient; and
drawing air from the protective volume while the face of the patient is in the protective volume.

20. The method of claim 19, further comprising:
coupling a vacuum source with the patient barrier device.
